(19) United States
(12) Patent Application Publication
(10) Pub. No.: US 2006/0110454 A1
(43) Pub. Date: May 25, 2006

(54) Extended release formulation of pramipexole dihydrochloride

(76) Inventors: Rajesh Kshirsagar, Vadodara (IN); Mayank Joshi, Vadodara (IN); Amresh Kumar, Vadodara (IN)

Correspondence Address: PENDORF & CUTLIFF, 5111 Memorial Highway, Tampa, FL, 33634-7356, US

(21) Appl. No.: 11/260,066

(22) Filed: Oct. 27, 2005

(30) Foreign Application Priority Data
Oct. 27, 2004 (IN) 1153/MUM/2004

(51) Int. Cl. A61K 31/428; A61K 9/22

(52) U.S. Cl. 424/468; 514/367

(57) ABSTRACT

An extended release composition of Pramipexole or a pharmaceutical acceptable salt thereof, wherein the active agent is coated on a non pareil inert core, the drug loaded core is further coated with a polymeric layer which enables the release of the active agent over an extended period and optionally the extended release pellets being further blended with suitable excipients and compressed into a multi unit tablet and processes for the preparation of the said composition.

### BACKGROUND OF THE INVENTION

1. Field of the Invention

The present invention relates to the process of preparing extended release formulation of Pramipexole. The formulation of the present invention is an extended release pellets. Pramipexole is a dopamine D2 receptor agonist useful in treatment of Parkinson's disease. Pramipexole as its dihydrochloride salt is commercially available as MIRAPEX tablets of Pharmacia & Upjohn. These are immediate-release tablets in 0.125 mg, 0.25 mg, 0.5 mg, 1.0 mg and 1.5 mg strengths, designed for oral administration of a single tablet three times per day to provide a daily dose of 0.375 to 4.5 mg. Doses herein are expressed in amounts of pramipexole dihydrochloride monohydrate unless otherwise specified; 1.0 mg pramipexole dihydrochloride monohydrate is equivalent to about 0.7 mg pramipexole base.

2. Related Art of the Invention

The chemical name of pramipexole dihydrochloride is (S)-2-amino-4,5,6,7-tetrahydro-6-(propylamino)benzothiazole dihydrochloride monohydrate (FIG. 1). Its empirical formula is C1OH17N3S.2 HCl.H2O, and its molecular weight is 302.27. Pramipexole dihydrochloride is a white to off-white powder substance. Pramipexole dihydrochloride is more than 20% soluble in water, about 8% in methanol, about 0.5% in ethanol, and practically insoluble in dichloromethane.

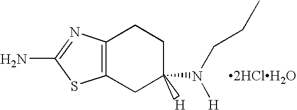

FIG. 1: Structure of Pramipexole Dihydrochloride.

The primary indication for the drug, Parkinson's disease, is an affliction that becomes more prevalent with advancing age and is often accompanied by decline in memory (elderly patients). Though a three times daily dosing regimen for immediate-release pramipexole dihydrochloride tablets is well tolerated, for enhancing patient compliance a once-daily regimen is explored in International patent applications, WO 2004010999, WO 2004010997 A1 and WO 04010982.

### SUMMARY OF THE INVENTION

The process covered under these patent applications involve the preparation of hydrophilic matrix tablet using hydroxypropyl methylcellulose (HPMC) as the rate controlling polymer and pregelatinized starch of a specific tensile strength as the filler. The tablet is prepared by the process of direct compression wherein all the ingredients except lubricant are blended first in a V-blender for 10 to 30 minutes at 24 rpm, lubricant is added to it and mixed for few minutes and finally the blend is compressed into tablet. The hydrophilic matrix tablet is further coated with a rate controlling ethyl cellulose (EC). The rate is also controlled by the formation of pores due to hydroxypropyl methylcellulose inside the diffusion layer of ethyl cellulose.

The prior art mentions that side-effect profile will be less with once daily dosage form compared to thrice daily immediate release dosage form. It identifies an in vitro release profile that would be characteristic of a well tolerated once-daily dosage form of pramipexole. It also provides an in-vivo pharmacokinetic (PK) profile that would be consistent with good therapeutic efficacy while not causing an unacceptable incidence or severity of side effects.

There are limitations of the prior art. These are
1. It involves a synergistic approach among HPMC and EC in obtaining extension of drug release. Therefore, for all the five strengths, a different composition is required to be derived for having the same dissolution profile. It does not show any possibility for having a step-up step-down composition. There is no correlation among the composition for all the strength. Although the composition is same qualitatively but is different quantivatitively. The percentage of ingredients in the final dosage form varies for all the five strengths.
2. Very low level of coating (3 to 5%) of the rate controlling polymer of the dosage form. So there is high possibility of having variation in the coating thickness, specifically at the edges of the tablet.
3. Extremely lower dose of 375 and 750 micrograms can pose a problem of content non-uniformity during compression of the tablets.

The dosage form in international patent application WO2004010999 provides an extended release product with the following probable in vitro dissolution specifications:

| Time | 1% | 2% | 3% |
|---|---|---|---|
| (h) | dissolved | dissolved | dissolved |
| 0 | 0 | 0 | 0 |
| 1 | 15 | 11 | 0 |
| 2 | 24 | 20 | 0.5 |
| 4 | 36 | 34 | 15 |
| 6 | 47 | 46 | 23 |
| 8 | 55 | 55 | 29.6 |
| 12 | 69 | 70 | 41.6 |
| 16 | 79 | 80 | 51.1 |
| 24 | 90 | 92 | 64.8 |

Protocol: USP Apparatus I, 900 ml 0.05M Phosphate Buffer pH 6.8, 100 rpm, 37° C.

In the present invention an alternative once daily extended release formulation is developed. It describes a process which overcomes the limitation of prior invention. The process of the earlier invention necessitates the development of a unique formula for all strengths, whereas present invention discloses step-up step-down composition, which adds to a very high degree of convenience to the fabricator of the product.

United state patent application No. 20050118264 discloses an extended release composition comprising as active compound Venlafaxine Hydrochloride, in which Venlafaxine Hydrochloride is coated on a non pareil inert core, which coated core is then coated with polymeric layer which enables the controlled release of the Venlafaxine Hydrochloride. The present invention comprises of pramipexole dihydrochloride, as active compound. Pramipexole dihydrochloride is low dose, highly photosensitive and characteristically different active than the venlafaxine hydrochloride. The process in the present invention provides uniform content and dose loading.

The present invention of extended release formulation comprising of pramipexole dihydrochloride and pharmaceutically acceptable excipients. Pramipexole dihydrochloride is coated on a non pareil inert core, said coated core is then coated with a polymeric layer which enables the controlled release of pramipexole dihydrochloride. Pramipexole dihydrochloride comprises 0.01 to 10.0% w/w of the coated pellets.

In a preferred embodiment of the present invention, pramipexole dihydrochloride is suitably admixed with binder, said binder is selected from polyvinyl pyrrolidone (povidone), hydroxypropyl cellulose, hydroxypropyl methylcellulose, etc. Binder preferably comprises 0.5 to 20% w/w of the coated pellets. The non-pareil inert core can be either inert sugar core or microcrystalline cellulose core or the equivalents thereof. The composition preferably comprises 10 to 90% of the core per weight of the coated pellets. Advantageously the coated core is then coated with an isolating layer (sub coating). Isolating (sub-coating) layer composed of polymers selected from polyvinyl pyrrolidone, hydroxypropyl methylcellulose, microcrystalline cellulose, Hydroxypropyl cellulose, carrageenan, glyceryl monostearate, etc. The sub coating layer comprises of 0.5 to 10% w/w of the coated pellets.

The sub-coating layer is then coated with an additional polymeric layer which enables the extended release of pramipexole dihydrochloride. Said additional polymeric layer composed of hydrophobic polymer, hydrophobic or hydrophilic plasticizer and/or hydrophilic pore forming polymer. Said additional polymeric layer is suitably sprayed over the coated non-pareil layer or over the sub-coating layer. The hydrophobic polymer used in said additional polymeric layer are polyvinyl acetate, eudragit, cellulose derivatives such as ethyl cellulose, cellulose acetate, etc. The hydrophilic pore forming polymers in said additional polymeric layer are copolyvidone, polyvinyl pyrrolidone, polyethylene glycols, hydroxylpropyl methyl cellulose, hydroxyethyl cellulose, etc. The plasticizer in said additional polymeric layer are dibutyl sebacate, triethyl citrate, castor oil, glyceryl monostearate, diethyl phthalate, glyceryl trihepthanoate, etc. The additional polymeric coating layer may also be wax based coating. The composition preferably comprises 2.0 to 60.0% of hydrophobic polymer per weight of the coated pellets; Nil to 25% per weight of hydrophillic pore forming polymer of the coated pellets and preferably Nil to 10% of plasticizer per weight of the coated pellets.

The above process is a conventional process and can be performed in fluidized bed coating system with preference to bottom spray mechanism. The pellets obtained are either suitably filled into hard gelatin capsules or compressed into tablets. The tablet if dispersible, will have suitable flavor. The tablet for swallowing may be coated with a non functional film coating; process is common to the person with limited skills in the art. When the small coated particles of pramipexole dihydrochloride are tabletted they are mixed with additives e.g. microcrystalline cellulose such as Avicel PH 102, Avicel PH 301, Avicel.RTM., which improves the tabletting properties and facilitates the disintegration of the tablet, whereby the individual beads are liberated

The present invention can be illustrated by the following examples without being limited by them.

### EXAMPLE 1 to 6

Pramipexole Dihydrochloride sustained release pellets were prepared having the composition shown in Table 1.

Drug Layering:

Hydroxypropyl Methylcellulose (3 cps) was dispersed in purified water and pramipexole dihydrochloride was disolved in the formed dispersion. This dispersion was coated on sugar spheres (700 micron) using a fluid bed coater.

Sub Coating:

Sub coating solution was prepared by dissolving povidoneK-30 in denatured ethanol. This solution was coated on drug loaded pellets using a fluid bed coater.

Functional Coating:

Functional coating solution was prepared by dispersing ethocel 45 cps in denatured ethanol. The polymer was allowed to hydrate for 10 hrs and form a clear dispersion. Dibutyl sebacate was added to the solution just 1 hour before coating and mixed well. Solution was coated on sub coated pellets using a fluid bed coater.

Table 1: Composition of Pramipexole Dihydrochloride Pellets of Example 1 to 6.

| | Quantity (mg) | | | | | |
|---|---|---|---|---|---|---|
| Ingradient | Example 1 | Example 2 | Example 3 | Exammple 4 | Example 5 | Example 6 |
| Drug layering | | | | | | |
| stage | | | | | | |
| Pramipexole | 0.375 | 0.375 | 0.375 | 0.375 | 0.375 | 0.375 |
| dihydrochloride | | | | | | |
| HPMC 3 cps | 37.5 | 37.5 | 37.5 | 37.5 | 37.5 | 37.5 |
| Purified water | qs | qs | qs | qs | qs | qs |
| Sugar spheres | 150 | 150 | 150 | 150 | 150 | 150 |
| Sub coating | | | | | | |
| stage | | | | | | |
| Povidone K 30 | 3.76 | 3.76 | 3.76 | 3.76 | 3.76 | 3.76 |
| Ethanol | 37.58 | 37.58 | 37.58 | 37.58 | 37.58 | 37.58 |
| Functional | | | | | | |
| coating stage | | | | | | |
| Ethyl cellulose | 8.52 | 13.63 | 17.03 | 20.44 | 23.85 | 30.66 |
| 45 cps | | | | | | |
| Ethanol | 319.4 | 511.04 | 638.8 | 766.56 | 894.32 | 1149.84 |
| Dibutyl | 0.95 | 1.52 | 1.89 | 2.27 | 2.65 | 3.41 |
| sebacate | | | | | | |
| Total | 201.11 | 206.79 | 210.56 | 214.35 | 218.14 | 225.71 |

### EXAMPLE 7

Dissolution profiles of the pramipexole dihydrochloride pellets of each of Examples 1 to 6 were evaluated under the following conditions. USP apparatus 1 was used to stir a dissolution medium (900 ml of phosphate buffer at a pH of 6.8) at a spindle rotation speed of 100 rpm and a temperature of 37° C. The dissolution rate was shown in Table 2.

Table 2: In Vitro Dissolution Data for Example 1 to 6.

| | % dissolved | | | | | |
|---|---|---|---|---|---|---|
| Time | Ex- | Ex- | | | | |
| (hr) | ample 1 | ample 2 | Example 3 | Example 4 | Example 5 | Example 6 |
| 0 | 0 | 0 | 0 | 0 | 0 | 0 |
| 1 | 31 | 14 | 13 | 11 | 9 | 5 |
| 2 | 53 | 30 | 26 | 23 | 19 | 14 |
| 4 | 72 | 53 | 49 | 44 | 39 | 31 |
| 6 | 83 | 66 | 62 | 57 | 51 | 44 |
| 8 | 89 | 73 | 70 | 66 | 60 | 53 |
| 12 | 93 | 81 | — | — | 70 | — |
| 24 | — | — | — | 87 | 84 | 80 |

### EXAMPLE 8

Multi-particulate tablets of Pramipexole dihydrochloride sustained release pellets were prepared having the composition shown in Table 3.

All ingredients except pramipexole dihydrochloride pellets and lubricants were screened to remove lumps and blended thoroughly for 30 minutes with Pramipexole dihydrochloride pellets using conta blender at 15 rpm. The screened lubricant was then blended with it for further 3-5 min. The resulting mixture was compressed.

Table 3: Composition of Pramipexole Dihydrochloride Multi-Particulate Tablets of

Example 8

| | Quantity |
|---|---|
| Ingredients | (mg) |
| Pramipexole dihydrochloride | 225.71 |
| pellets (Example 6) | |
| Talc | 0.75 |
| Micro crystalline cellulose PH | 486.85 |
| 301 | |
| Micro crystalline cellulose PH | 243.69 |
| 102 | |
| Cross carmellose sodium | 18.00 |
| Aerosil 200 | 1.5 |
| Sodium stearyl fumerate | 1.5 |
| Total | 978.0 |

### EXAMPLE 9

Pramipexole Dihydrochloride sustained release pellets were prepared having the composition shown in Table 4.

Drug Layering:

Hydroxypropyl Methylcellulose (3 cps) was dispersed in purified water and pramipexole dihydrochloride was disolved in the formed dispersion. This dispersion was coated on microcrystalline cellulose beads (500-710 micron) using a fluid bed coater.

Functional Coating:

Functional coating solution was prepared by dispersing Surelease E7 19010 in purified water. The polymer was allowed to mix for 60 minutes and form a uniform dispersion. Solution was coated on sub coated pellets using a fluid bed coater.

### EXAMPLE 10

Pramipexole Dihydrochloride sustained release pellets were prepared having the composition shown in Table 4.

Drug Layering:

Povidone K30 was dispersed in purified water and pramipexole dihydrochloride was disolved in the formed dispersion. This dispersion was coated on microcrystalline cellulose beads (500-710 micron) using a fluid bed coater.

Sub Coating:

Sub coating solution was prepared by dissolving povidone K30 in purified water. This solution was coated on drug loaded pellets using a fluid bed coater.

Functional Coating:

Functional coating solution was prepared by dispersing Surelease E7 19010 in purified water. The polymer was allowed to mix for 60 minutes and form a uniform dispersion. Solution was coated on sub coated pellets using a fluid bed coater.

Table 4: Composition of Pramipexole Dihydrochloride Pellets of Example 9-11.

| | Quantity (mg) | | |
|---|---|---|---|
| Ingradient | Example 9 | Example 10 | Example 11 |
| Drug layering stage | | | |
| Pramipexole | 3.0 | 3.0 | 3.0 |
| dihydrochloride | | | |
| HPMC 3 cps | 3.0 | | 21.0 |
| Povidone K 30 | | 6.0 | |
| Purified water | qs | qs | qs |
| Celphere CP 203 | — | — | 263 |
| Celphere CP 507 | 281 | 275 | — |
| Sub coating stage | | | |
| HPMC 3 cps | — | — | 8.0 |
| Povidone K 30 | — | 3.0 | — |
| Purified water | — | qs | qs |
| Functional coating | | | |
| stage | | | |
| Surelease E7 19010 | 25.83 | 23.0 | — |
| Ethocel 45 cps | — | — | 17.7 |
| HPMC 3 cps | — | — | 4.42 |
| Purified water | qs | qs | — |
| Denatured ethanol | — | — | qs |
| Total | 312.83 | 310.0 | 317.12 |

### EXAMPLE 11

Pramipexole Dihydrochloride sustained release pellets were prepared having the composition shown in Table 4.

Drug Layering:

Hydroxypropyl Methylcellulose (3 cps) was dispersed in purified water and pramipexole dihydrochloride was disolved in the formed dispersion. This dispersion was coated on microcrystalline cellulose beads (150-300 micron) using a fluid bed coater.

Sub Coating:

Sub coating solution was prepared by dissolving Hydroxypropyl Methylcellulose (3 cps) in purified water. This solution was coated on drug loaded pellets using a fluid bed coater.

Functional Coating:

Functional coating solution was prepared by dispersing ethocel 45 cps in denatured ethanol. The polymer was allowed to hydrate for 10 hrs and form a clear dispersion. Hydroxypropyl Methylcellulose (3 cps) was added to the solution and allowed to hydrate to form the clear solution. Solution was coated on sub coated pellets using a fluid bed coater.

### EXAMPLE 12

Pramipexole Dihydrochloride sustained release pellets were prepared having the composition shown in Table 5.

Drug Layering:

Povidone K30 was dispersed in purified water and pramipexole dihydrochloride was disolved in the formed dispersion. This dispersion was coated on microcrystalline cellulose beads (500-710 micron) using a fluid bed coater.

Sub Coating:

Sub coating solution was prepared by dissolving Povidone K30 in purified water. This solution was coated on drug loaded pellets using a fluid bed coater.

Functional Coating:

Functional coating solution was prepared by dispersing ethocel 7 cps in denatured ethanol. The polymer was allowed to hydrate for 10 hrs and form a clear dispersion. Povidone K30 was added to the solution and allowed to hydrate to form the clear solution. Solution was coated on sub coated pellets using a fluid bed coater.

Table 5: Composition of Pramipexole Dihydrochloride Pellets of Example 12.

| | Quantity |
|---|---|
| Ingradient | (mg) |
| Drug layering stage | |
| Pramipexole | 3.0 |
| dihydrochloride | |
| Povidone K 30 | 24 |
| Purified water | qs |
| Celphere CP 507 | 275 |
| Sub coating stage | |
| Povidone K 30 | 9 |
| Purified water | Qs |
| Functional coating | |
| stage | |
| Ethocel 7 cps | 28 |
| Povidone K30 | 3.11 |
| Denatured ethanol | Qs |
| Total | 342.1 |

### EXAMPLE 13

Dissolution profiles of the pramipexole dihydrochloride pellets of each of Examples 8 to 12 were evaluated under the following conditions. USP apparatus 1 was used to stir a dissolution medium (900 ml of phosphate buffer at a pH of 6.8) at a spindle rotation speed of 100 rpm and a temperature of 37° C. The dissolution rate was shown in Table 6.

Table 6: In Vitro Dissolution Data for Example 8 to 12.

| Time | % dissolved | | | | |
|---|---|---|---|---|---|
| (hr) | Example 8 | Example 9 | Example 10 | Example 11 | Example 12 |
| 0 | 0 | 0 | 0 | 0 | 0 |
| 1 | 22 | 6 | 12 | 16 | 18 |
| 2 | 34 | 11 | 21 | 28 | 32 |
| 4 | 50 | 24 | 37 | 41 | 50 |
| 6 | 61 | 39 | 48 | 48 | 62 |
| 8 | 69 | 53 | 56 | 54 | 68 |
| 12 | 78 | 73 | 67 | 61 | 77 |
| 16 | 84 | 83 | 74 | 66 | 82 |
| 24 | 91 | 91 | 83 | 71 | 87 |

1. An extended release formulation of Pramipexole or a pharmaceutical acceptable salt thereof as active agent, in which
active agent is coated on a nonpareil inert core,
the active agent loaded core is further coated with a polymeric layer which enables the release of the active agent over an extended period to produce extended release pellets, and
optionally the extended release pellets are further blended with suitable excipients and compressed into a multi-unit tablet.

2. An extended release formulation according to claim 1, where in said salt is Pramipexole dihydrochloride.

3. An extended release formulation according to claim 1, wherein the formulation comprises 0.01-10% of Pramipexole dihydrochloride per weight of the total dosage form.

4. An extended release formulation according to claim 1, wherein the formulation comprises about 0.125 to about 6 mg pramipexole, expressed as pramipexole dihydrochloride monohydrate equivalent, per dosage unit.

5. An extended release formulation of Pramipexole or a pharmaceutical acceptable salt thereof, having the following dissolution profile in USP Apparatus 1 (basket) at 100 rpm in Phosphate Buffer pH 6.8 at 37 degree. C.:

| Time (hours) | Average % Pramipexole released |
|---|---|
| 1 | <25 |
| 6 | 30-60 |
| 12 | 55-75 |
| 24 | >80 |

6. An extended release formulation according to claim 2, wherein the Pramipexole dihydrochloride is suitably admixed with binder.

7. An extended release formulation according to claim 6, wherein the formulation comprises 0.5%-20% of the binder per weight of the total dosage form.

8. An extended release formulation according to claim 7, wherein the binder is selected among polyvinyl pyrrolidone (povidone), hydroxypropyl cellulose and hydroxypropyl methylcellulose.

9. An extended release formulation according to claim 1, which comprises 10-90% of the nonpareil core per weight of the total dosage form.

10. An extended release formulation according to claim 9, wherein the nonpareil inert core can be either inert sugar core, silicon dioxide or microcrystalline cellulose core or the equivalents thereof.

11. An extended release formulation according to claim 10, wherein the nonpareil inert cores have a size of 0.1-1.0 mm.

12. An extended release formulation according to claim 1, wherein the core and/or the core coated with pramipexole dihydrochloride is coated with an insulating/protecting layer composed of polymers selected from polyvinyl pyrrolidone, hydroxypropyl methylcellulose, microcrystalline cellulose, Hydroxypropyl cellulose, carrageenan and glyceryl monostearate.

13. An extended release formulation according to claim 12, wherein the insulating layer is comprised of 0.5-10% of the insulating layer per weight of the total dosage form.

14. An extended release formulation according to claim 1, wherein the extended release polymeric layer is composed, e.g. of a hydrophobic polymer, hydrophobic or hydrophilic plasticizer and/or hydrophilic release modulator polymer.

15. An extended release formulation according to claim 1, which comprises 2-60% of the hydrophobic polymer per weight of the total dosage form, optionally up to 25% of the hydrophillic release modulator polymer per weight of the total dosage form and/or optionally up to 20% of the plasticizer per weight of the total dosage form.

16. An extended release formulation according to claim 15, wherein said hydrophobic coating polymers are selected among polyvinyl acetate, eudragit, cellulose derivatives such as ethyl cellulose, cellulose acetate and their plasticizers are selected among dibutyl sebacate, triethyl citrate, castor oil, glyceryl monostearate, diethyl phthalate, glyceryl trihepthanoate.

17. An extended release formulation according to claim 15, wherein the hydrophilic release modulator polymer is selected among copolyvidone, polyvinyl pyrrolidone, polyethylene glycols, hydroxylpropyl methyl cellulose and hydroxyethyl cellulose.

18. An extended release formulation according to claim 1, wherein said formulation is filled into hard gelatin capsules.

19. An extended release formulation comprising the extended release preparation according to claim 1 and pharmaceutical additives compressed to tablets which disintegrate to release the preparation when the tablets are brought into contact with gastrointestinal fluids.

20. A method for preparing an extended release formulation of Pramipexole or a pharmaceutical acceptable salt thereof as active agent, comprising the steps of:
I. dissolving Pramipexole dihydrochloride and binder in a suitable solvent system to prepare a clear solution;
II. applying a coat thereof to a nonpareil inert core with above solution using a fluid bed processor;
III. further coating the active agent loaded core with an isolating/protecting coat;
IV. further coating the above core with a polymeric layer which enables the release of the active agent over an extended period;
V. filling of extended release pellets into hard gelatin capsules; and
VI. optionally blending the pellets with suitable excipients and compressing into a multi unit tablet.
